(19) United States
(12) Patent Application Publication
(10) Pub. No.: US 2007/0258885 A1
(43) Pub. Date: Nov. 8, 2007

(54) Device for concentrating Technetium-99m pertechnetate and method thereof

(76) Inventors: Tsai-Yueh Luo, Lung-Tan (TW); Ai-Ren Lo, Lung-Tan (TW); Wuu-Jyh Lin, Lung-Tan (TW); Tseng-Chung Huang, Lung-Tan (TW); Bor-Tsung Hsieh, Taichung (TW); Ching-Jun Liou, Lung-Tan (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/429,194

(22) Filed: May 8, 2006

(51) Int. Cl. A61N 5/00; A61K 51/00

(52) U.S. Cl. 424/1.11; 600/1

(57) ABSTRACT

A device for concentrating ^{99m}Tc pertechnetate and a method thereof are disclosed. The device includes a concentration device, a control device and a central processing unit. The concentration device is for concentrating ^{99m}Tc pertechnetate, the control device connects with each members of the concentration device, and the central processing unit is used for saving an automatic control program. The automatic control program is run by the central processing unit so as to detect and monitor weight as well as activity of the concentrated ^{99m}Tc pertechnetate. Due to the automatical control, the concentration quality and production efficiency of the ^{99m}Tc pertechnetate are improved. Moreover, the radiation dose received by users is reduced.

### BACKGROUND OF THE INVENTION

The present invention relates to a device for concentrating radioactive materials and a method thereof, especially to a device for concentrating ^{99m}Tc pertechnetate and a method thereof.

According to statistics made by Tzen, Kai-Yuan, director of nuclear medicine department, National Taiwan University Hospital on 2003, there are totally 88 SPECT (Single photon emission computed tomography) machines in nuclear medicine department of 43 hospitals in Taiwan. SPECT has been applied mainly to many fields of disease diagnosis, especially myocardial perfusion imaging and bone scan and the radioactive nuclide being mostly used is ^{99m}Tc. The result of this study is similar with the result of foreign studies. Taking data of American in 2002 as an example, the tests using radionucleotide ^{99m}Tc is as high as eleven million and fifty nine hundred thousand times, which occupied 80.9 percent of the entire tests. The data shows the importance of ^{99m}Tc in nuclear medicine.

Besides nuclide characteristics, ⁹⁹Mo/^{99m}Tc generator has the features of convenience, safety and easy operation that lead to its popularity in clinical use. However, after being used for a period of time, the specific activity is too low to be applied in clinical tests. Therefore, the old generators need to be replaced after a period of time. Technetium-99m is one of the most important radioisotopes used for medical diagnostics. It's formed from the decay of Molybdenum-99 which has a half-life of 67 hours. The characteristics of short half life and 140 keV gamma ray emission make ^{99m}Tc as an ideal for SPECT in clinical nuclear medicine imaging procedures or detection of tumor cells, such as ^{99m}Tc-MDP bone imaging, ^{99m}Tc-methoxyisobutylisonitrile (MIBI) SPECT in the detection of breast cancer, ^{99m}Tc myoview for myocardial imaging, ^{99m}Tc-HMPAO and ^{99m}Tc-ECD for SPECT imaging of cerebral blood flow, ^{99m}Tc-MAG3 for renal function scintigraphy and ^{99m}Tc-TRODAT-1 imaging for diagnosis of Parkinson's disease, and so on. These show the importance and the potential of this diagnostic nuclide-^{99m}Tc in nuclear medicine.

Technetium-99m solution is obtained from ⁹⁹Mo/^{99m}Tc generator. Firstly get Technetium-99m pertechnetate by elution of with 12 ml normal saline. Then concentrate Technetium-99m pertechnetate by means of continuous bi-column solid chromatography, absorb Technetium-99m pertechnetate by anion exchange resin, and elute Technetium-99m pertechnetate by normal saline. Collect the eluant in different stages to manufacture Technetium-99m pertechnetate for medical use.

### SUMMARY OF THE INVENTION

Therefore it is a primary object of the present invention to provide a device for concentrating ^{99m}Tc pertechnetate and a method thereof that remotely monitors and in-time shows the reaction processing of condensation by automatic control so that the concentration quality and production efficiency of the ^{99m} Tc pertechnetate are improved.

It is another primary object of the present invention to provide a concentration device for ^{99m} Tc pertechnetate and method thereof that makes reaction happen inside a close space so as to avoid exposure to radioactive material and reduce the radiation dose received by the operators. Since the radioactive nuclide enters the close system through pipelines for being condensed until the final products are output, the whole reaction is happened inside the close space. In order to achieve objects, the present invention reveals a concentration device for ^{99m}Tc pertechnetate and method thereof that controls concentration process of the ^{99m}Tc pertechnetate by means of automatic control program. The device consists of a concentration unit and a control unit. The ^{99m}Tc pertechnetate is concentrated through a cation-exchange solid phase extraction chromatography column and an anion exchange column inside the concentration device and the activity of the ^{99m}Tc pertechnetate inside a receiving flask is measured by a radiation measurement module of the control device that connects to a Geiger-Muller Counter. Moreover, a weighting scale member of a signal measurement module is used to weight the receiving flask.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein

FIG. 1A is a block diagram showing structure of an embodiment in accordance with the present invention;

FIG. 1B is a block diagram showing a concentration device of an embodiment in accordance with the present invention;

FIG. 1C is a block diagram showing a control device of an embodiment in accordance with the present invention;

FIG. 1D is a block diagram showing a control device of an embodiment in accordance with the present invention;

FIG. 2 is a flow chart showing concentration process of an embodiment in accordance with the present invention;

FIG. 3 is a block diagram showing a concentration device of another embodiment in accordance with the present invention;

FIG. 4 is a flow chart showing concentration process of another embodiment in accordance with the present invention;

FIG. 5 is a block diagram showing a concentration device of a further embodiment in accordance with the present invention;

FIG. 6 is a flow chart showing concentration process of a further embodiment in accordance with the present invention;

FIG. 7 is a block diagram showing a concentration device of a further embodiment in accordance with the present invention;

FIG. 8 is a schematic drawing showing measurement of Tc-99m activity vs time of a ⁹⁹Mo/^{99m}Tc generator in accordance with the present invention;

FIG. 9 is a schematic drawing showing measurement of ⁹⁹Mo activity vs time of a ⁹⁹Mo/^{99m}Tc generator in accordance with the present invention.

### DETAILED DESCRIPTION OF THE PREFERRED EMBODIMENT

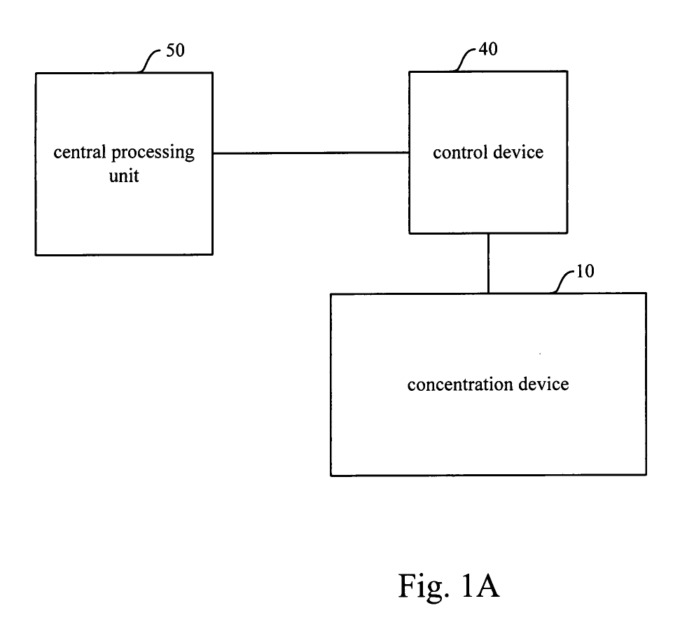

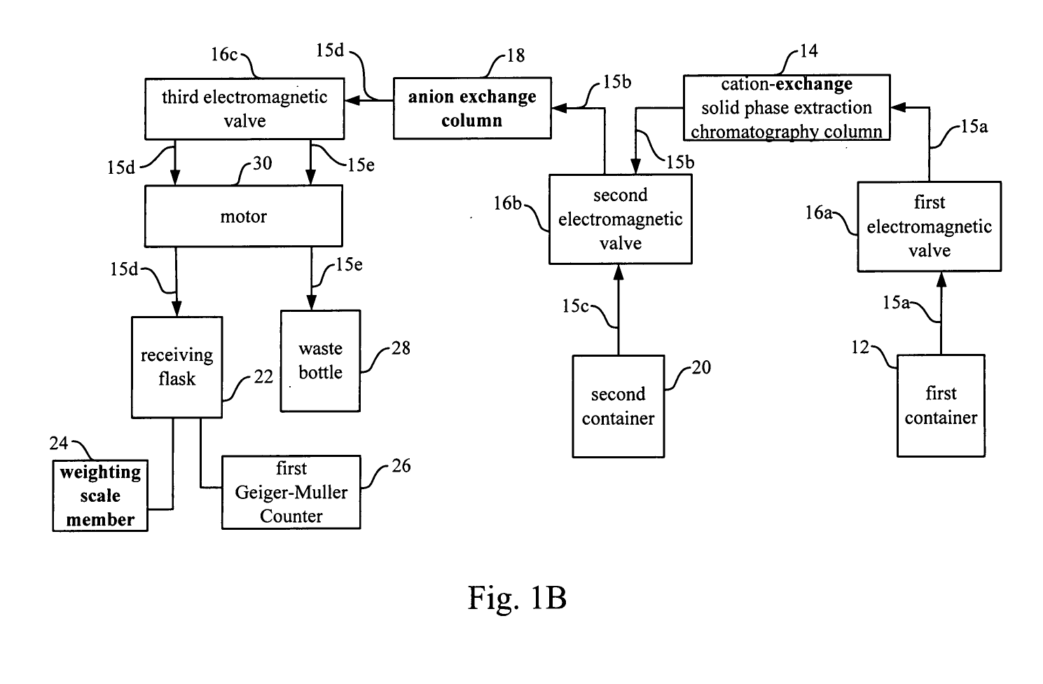

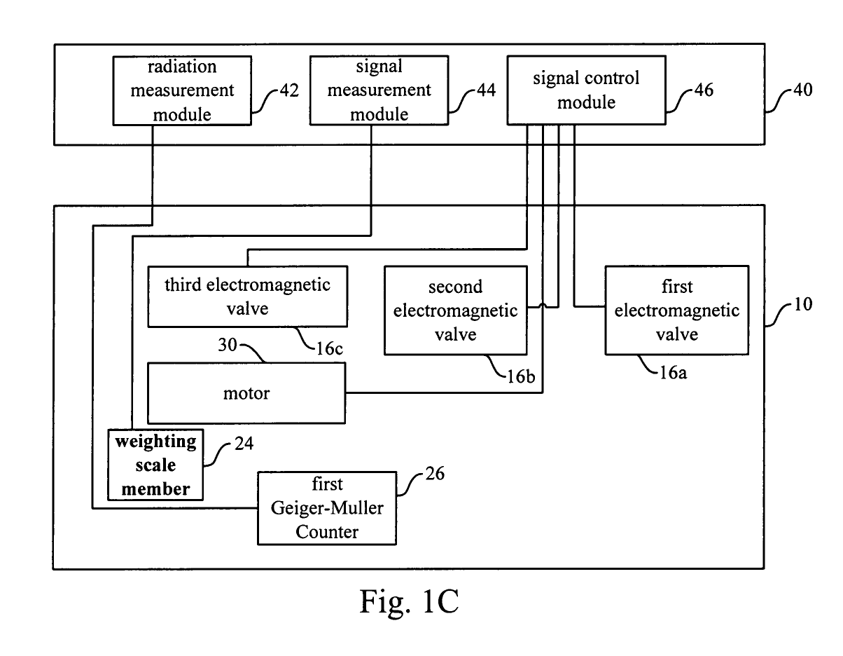

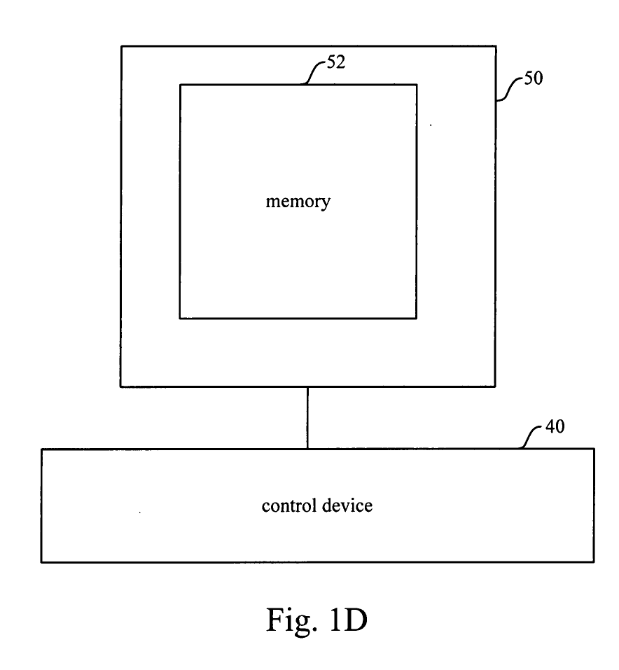

Refer to FIG. 1A, the present invention includes a concentration device 10, a control device 40 and a central processing unit (CPU) 50. The concentration device 10 is for carrying out concentration reaction while the control device 40 is for automatic control of the concentration device 10 by means of the central processing unit 50 that executes the automatic control program to run certain procedures for concentrating the ^{99m}Tc pertechnetate (technetium-99m pertechnetate). For example, the volume of the ^{99m}Tc pertechnetate is reduced from 12 ml to 1 ml.

Refer to FIG. 1B, the concentration device 10 in accordance with the present invention consists of a first container 12 for containing the ^{99m}Tc pertechnetate that is obtained by elution with normal saline from the ⁹⁹Mo/^{99m}Tc generator, a cation-exchange solid phase extraction chromatography column 14 that connects with the first container 12 by a first pipeline 15a and the first pipeline 15a having a first electromagnetic valve 16a, an anion exchange column 18 that connects with the cation-exchange solid phase extraction chromatography column 14 by a second pipeline 15b and the second pipeline 15b having a second electromagnetic valve 16b.

A second container 20 is for containing normal saline solution and is connected with the second electromagnetic valve 16b by a third pipeline 15c. A receiving flask 2 connects with the anion exchange column 18 by a fourth pipeline 15d while the fourth pipeline 15d includes a third electromagnetic valve 16c. A weighting scale member 24 and a first Geiger-Muller Counter 26 are disposed under the receiving flask 22 for detecting and monitoring the weight as well as the activity of the ^{99m}Tc pertechnetate therein. Furthermore, a waste bottle 28 is connected with the third electromagnetic valve 16c by a fifth pipeline 15e and a receiving flask 22 includes a first receiving flask and a second receiving flask. A motor 30—a creeping motor disposed between the fourth pipeline 15d and the fifth pipeline 15e transports the ^{99m}Tc pertechnetate or normal saline into the receiving flask 22 or the waste bottle 28. The cation-exchange solid phase extraction chromatography column 14 is a silver ion solid phase extraction chromatography column while the anion exchange column 18 is a SepPak anion exchange column. Lead is disposed around the first Geiger-Muller Counter 26 for warding off radioactive interference from the outside.

Refer to FIG. 1C, a control device 40 of an embodiment in accordance with the present invention is composed of a radiation measurement module 42, a signal measurement module 44, and a signal control module 46. The radiation measurement module 42 is connected with the first Geiger-Muller Counter 26 while the signal measurement module 44 is joined with the weighting scale member 24. And the signal control module 46 connects to the motor 30, the first electromagnetic valve 16a, the second electromagnetic valve 16b, and the third electromagnetic valve 16c. By the first Geiger-Muller Counter 26, the radiation measurement module 42 detects activity of the concentrated ^{99m}Tc pertechnetate inside the receiving flask 22. By the weighting scale member 24, the signal measurement module 44 gets weight of the concentrated ^{99m}Tc pertechnetate inside the receiving flask 22.

Refer to FIG. 1D, the central processing unit (CPU) 50 according to the present invention includes a memory 52 for saving the automatic control program and connects to the control device 40. The central processing unit 50 executes the automatic control program.

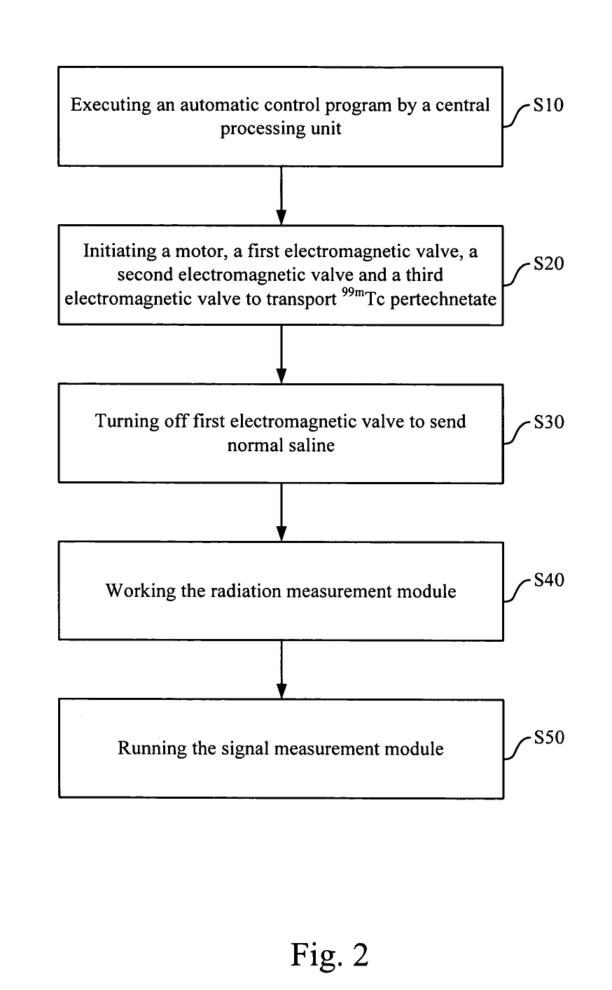

Refer to FIG. 2, a flow chart of an embodiment of a method for concentrating ^{99m}Tc pertechnetate in accordance with the present invention is disclosed. Refer to step S10, an automatic control program is executed by a central processing unit (CPU) 50 so as to drive a radiation measurement module 42, a signal measurement module 44, and a signal control module 46. In step S20, initiate a motor 30, a first electromagnetic valve 16a, a second electromagnetic valve 16b, and a third electromagnetic valve 16c so that ^{99m}Tc pertechnetate in a first container 12 is transported into a cation-exchange solid phase extraction chromatography column 14, an anion exchange column 18 and waste bottle 28 through a first pipeline 15a, a second pipeline 15b, a fourth pipeline 15d, and a fifth pipeline 15e. Refer to step S30, the first electromagnetic valve 16a is turned off and normal saline inside a second container 20 is sent into the anion exchange column 18 and a first receiving bottle. Then the radiation measurement module 42 works to monitor activity of the ^{99m}Tc pertechnetate through a first Geiger-Muller Counter 26, as shown in step S40. In step S50, run the signal measurement module 44 to weight the ^{99m}Tc pertechnetate inside the first receiving flask by a weighting scale member 24 so as to check whether to interrupt the automatic control program or not.

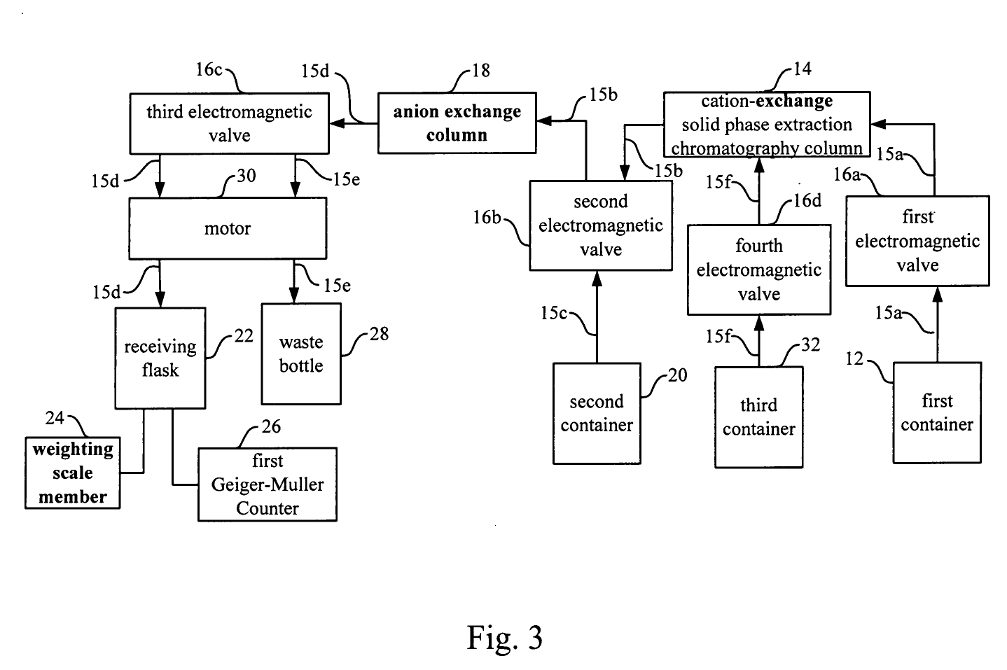

Refer to FIG. 3, an embodiment in this figure is different from the embodiment in FIG. 1B is in that the cation-exchange solid phase extraction chromatography column 14 in FIG. 1B connects with a first pipeline 15a and a first electromagnetic valve 16a while the cation-exchange solid phase extraction chromatography column 14 in FIG. 3 further connects with a sixth pipeline 15f and a fourth electromagnetic valve 16d. The fourth electromagnetic valve 16d connects to a third container 32 that contains normal saline Before the concentration device 10 running concentration procedures, the cation-exchange solid phase extraction chromatography column 14 needs to be washed so as to avoid influence on solid phase extraction chromatography of the ^{99m}Tc pertechnetate in the first container 12. Once the fourth electromagnetic valve 16d is turned on, the motor 30 transports normal saline in the third container 32 passing through the cation-exchange solid phase extraction chromatography column 14 for washing it and then through the anion exchange column 18, the fourth pipeline 15d, the fifth pipeline 15e and conveyed to the waste bottle 28.

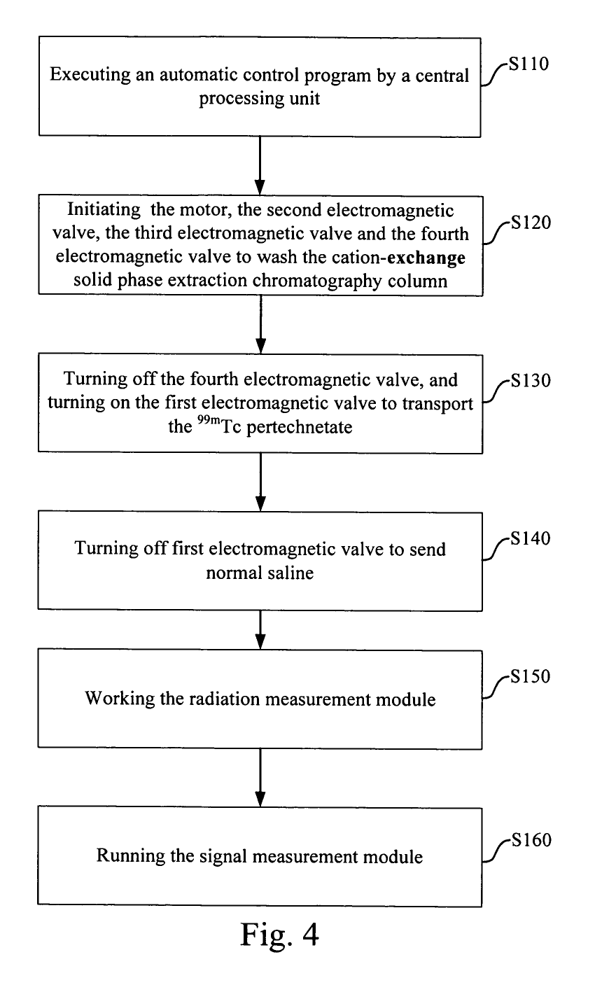

Refer to FIG. 4, the difference between the flow chart of the embodiment in this figure and the embodiment in FIG. 2 is in that this embodiment includes a further step-washing the cation-exchange solid phase extraction chromatography column 14. A method for concentrating ^{99m}Tc pertechnetate in accordance with the present invention further includes a step S120—initiate the motor 30, the second electromagnetic valve 16b, the third electromagnetic valve 16c and the fourth electromagnetic valve 16d and wash the cation-exchange solid phase extraction chromatography column 14, as well as step S130—turn off the fourth electromagnetic valve 16d, turn on the first electromagnetic valve 16a and transport the ^{99m}Tc pertechnetate.

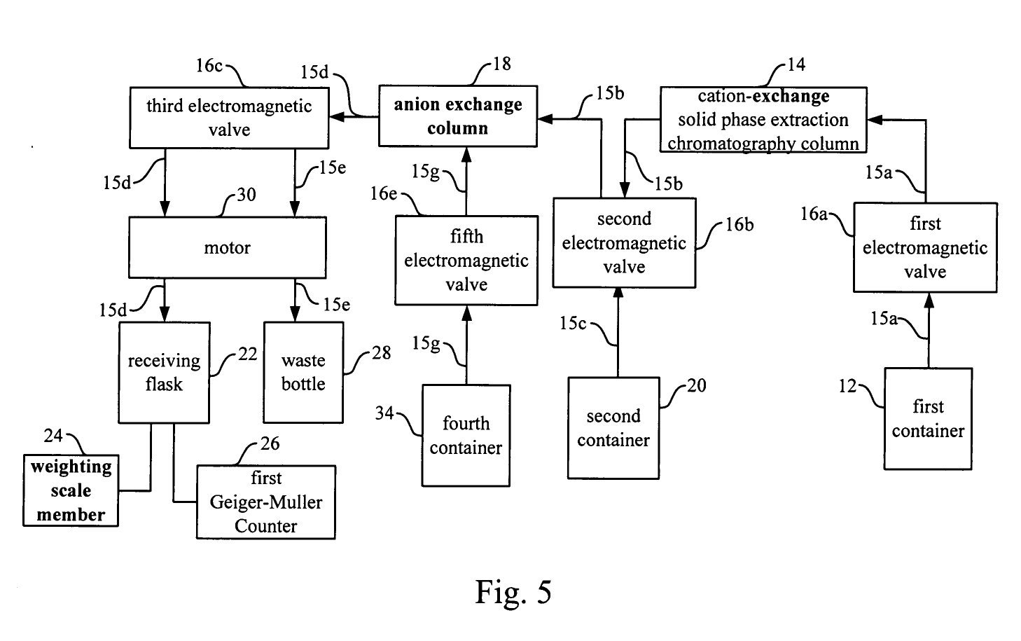

Refer to FIG. 5, the difference between the embodiment in this figure and the embodiment in FIG. 1B is in that the anion exchange column 18 in FIG. 1B connects with a first electromagnetic valve 16a, a second electromagnetic valve 16b, a first pipeline 15a and a second pipeline 15b while the anion exchange column 18 in FIG. 5 further connects with a seventh pipeline 15g and a fifth electromagnetic valve 16e. The fifth electromagnetic valve 16e connects to a fourth container 34 for containing normal saline. Once the fifth electromagnetic valve 16e is turned on, the motor 30 transports normal saline in the fourth container 34 into the anion exchange column 18 for eluting it and then the eluant is sent to the fourth pipeline 15d and the second receiving flask.

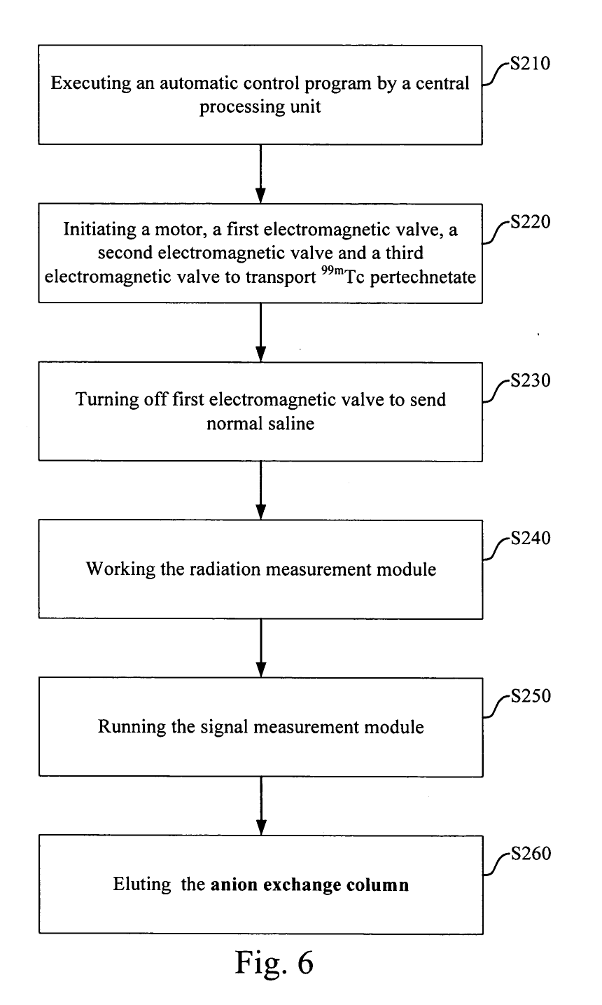

Refer to FIG. 6, the difference between the flow chart of the embodiment in this figure and the embodiment in FIG. 2 is in that this embodiment includes a further step-elute the anion exchange column 18. A method for concentrating ^{99m}Tc pertechnetate in accordance with the present invention further includes a step S260—elute the anion exchange column 18. Firstly replace the first receiving flask by the second receiving flask. Then turned off the second electromagnetic valve 16 band initiate the fifth electromagnetic valve 16e. The normal saline inside the fourth container 34 elutes the anion exchange column 18 for being sampled by the second receiving flask.

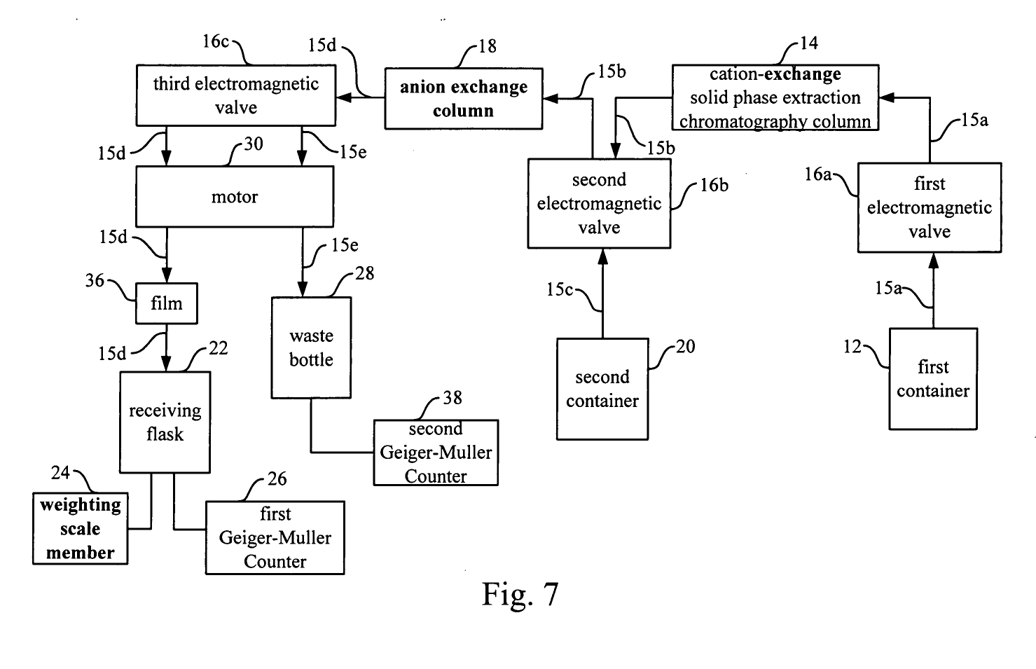

Refer to FIG. 7, the difference between the embodiment in this figure and the embodiment in FIG. 1B is in that a film 36 is disposed on top of the receiving flask 22 while a second Geiger-Muller Counter 38 is arranged on bottom of the waste bottle 28. And the film 36 of the concentration device 10 of the present invention is used to filter concentrated ^{99m}Tc pertechnetate obtained from normal saline and the second Geiger-Muller Counter 38 is used for the radiation measurement module 42 of the control device 40 to detect activity of the solution inside the waste bottle 28. If the solution inside the waste bottle 28 still contains the required level of activity, it can be recycled and concentrated again for being applied to the next test so as to reduce the waste.

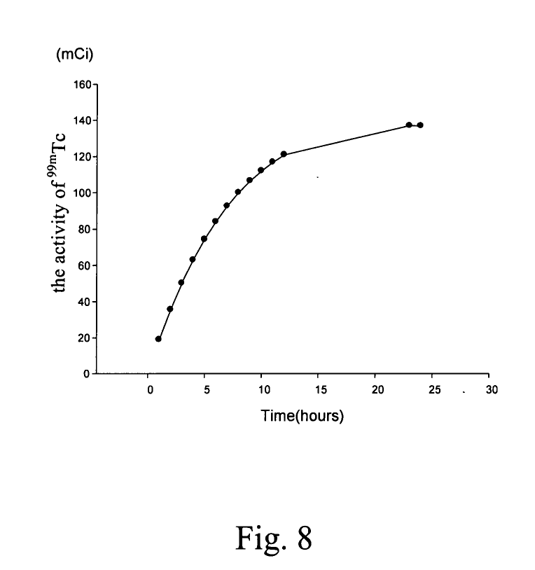

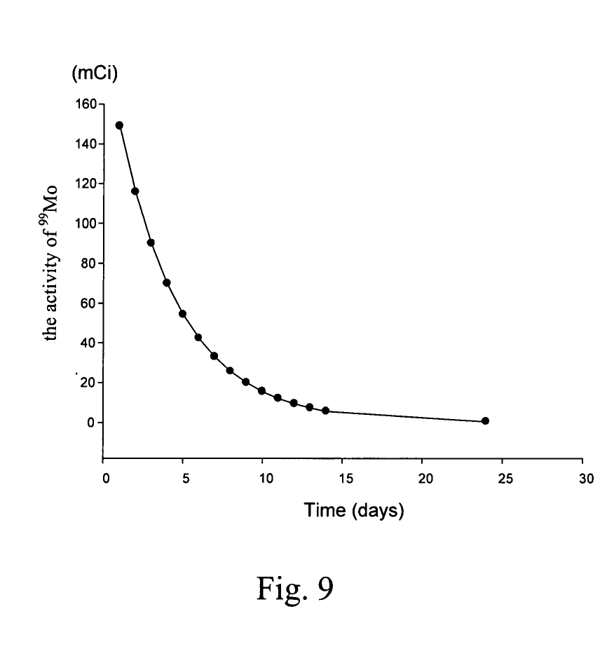

Therefore, it is learned that the concentration device of the present invention can prolong service life of generators. Take a ⁹⁹Mo/^{99m}Tc generator with activity of 200 mCi(minicurie) as an example, after the first elution, it takes 24 hours to get the maximum activity 140 mCi. And the activity of Tc-99m reduces along with the decay of parent nuclide ⁹⁹Mo, as shown in FIG. 8 & FIG. 9. After elution, the ⁹⁹Mo/^{99m}Tc generator is eluted again by 10 ml normal saline in the second hour. Then specific activity of the obtained Tc-99m is 35 mCi/10 ml.

The activity of above mentioned Tc-99m is not enough for clinical use on lots tests of nuclear medicine. For example, Tc-99m MIBI(Carolite) sold by pharmaceutical company—Bristol-Myers Squibb requires radioactivity level of 25-150 mCi/1-3 ml. The activity of recently released dopamine transporter imaging agent (^{99m}Tc TRODAT-1) is 6-8 mCi/ml. By means of a concentration device according to the present invention, activity of the Tc-99m is not reduced while the volume is reduced into 1 ml. Therefore, the efficiency of the ⁹⁹Mo/^{99m}Tc generator is dramatically improved.

Moreover, if the radioactivity of the ⁹⁹Mo/^{99m}Tc generator is 200 mCi, the radioactivity of the ⁹⁹Mo/^{99m}Tc is only 33 mCi for being eluted seven days. If the initial activity of the generator is 500 mCi, the radioactivity of the ⁹⁹Mo/^{99m}Tc at elapsed time of 11 days is only 30 mCi. As for hospitals, the specific activity is too low for clinical use and it need to purchase new generators. However, the present invention makes the radioactivity of ⁹⁹Mo/^{99m}Tc change from 30 mCi/10 ml to 30 mCi/1 ml. There is no need to replace old generators for preparation of radioisotopes. Furthermore, lifetime of each generator is extended at least 3 days.

The present invention is advantageous to prepare radiopharmaceuticals in clinical use, not only extends lifetime of generators, but also reduces cost for preparing radiopharmaceuticals. Moreover, due to automation of the concentration device of the present invention, the present invention decreases exposure time to radioactive material and further reduces the radiation dose to the operators. The present invention can also be applied to deal with concentration for Re-188 radioactive solution.

Additional advantages and modifications will readily occur to those skilled in the art. Therefore, the invention in its broader aspects is not limited to the specific details, and representative devices shown and described herein. Accordingly, various modifications may be made without departing from the spirit or scope of the general inventive concept as defined by the appended claims and their equivalents.

What is claimed is:

1. A device for concentrating ^{99m}Tc pertechnetate comprising:
a concentration device that having:
a first container for containing ^{99m}Tc pertechnetate;
a cation-exchange solid phase extraction chromatography column connecting with the first container by a first pipeline and the first pipeline having a first electromagnetic valve;
an anion exchange column connecting with the cation-exchange solid phase extraction chromatography column by a second pipeline and the second pipeline having a second electromagnetic valve;
a second container for containing normal saline solution connecting with the second electromagnetic valve by a third pipeline; and
a receiving flask connecting with the anion exchange column by a fourth pipeline and the fourth pipeline having a third electromagnetic valve; a weighting scale member and a first Geiger-Muller Counter are disposed under the receiving flask for detecting and monitoring weight and activity of the ^{99m}Tc pertechnetate inside the receiving flask;
a waste bottle connecting with the third electromagnetic valve by a fifth pipeline;
wherein the ^{99m}Tc pertechnetate or normal saline is transported into the receiving flask or waste bottle of the first pipeline, the second pipeline, the third pipeline, the fourth pipeline, and the fifth pipeline;
a control device having a radiation measurement module, a signal measurement module, and a signal control module; the radiation measurement module is connected with the first Geiger-Muller Counter and the signal measurement module is joined with the weighting scale member; the signal control module connects to the motor, the first electromagnetic valve, the second electromagnetic valve, and the third electromagnetic valve; and
a central processing unit having a memory for saving an automatic control program and connecting to the control device;
wherein the automatic control program is executed by the central processing unit for control of weight and activity of the ^{99m}Tc pertechnetate inside the receiving flask so as to concentrate the ^{99M}Tc pertechnetate automatically.

2. The device as claimed in claim 1, wherein a second Geiger-Muller Counter that connects with the radiation measurement module is disposed under the waste bottle for monitoring the activity of the ^{99m}Tc pertechnetate inside the receiving flask.

3. The device as claimed in claim 1, wherein a film is arranged on top of the receiving flask.

4. The device as claimed in claim 1, wherein the motor is a creeping motor and is disposed between the fourth pipeline and the fifth pipeline.

5. The device as claimed in claim 1, wherein the cation-exchange solid phase extraction chromatography column is a silver ion solid phase extraction chromatography column.

6. The device as claimed in claim 1, wherein the anion exchange column is a SepPak anion exchange column.

7. The device as claimed in claim 1, wherein the device further comprising a third container for containing sterilized water and the third container is connected with the cation-exchange solid phase extraction chromatography column through a sixth pipeline and a fourth electromagnetic valve.

8. The device as claimed in claim 1, wherein the device further comprising a fourth container for containing normal saline and the fourth container is connected with the second pipeline through a seventh pipeline and a fifth electromagnetic valve.

9. The device as claimed in claim 1, wherein lead is disposed around Lead is disposed around the first Geiger-Muller Counter and the second Geiger-Muller Counter for shielding radioactive interference from the outside.

10. A method for concentrating ^{99m}Tc pertechnetate comprising the steps of:
executing an automatic control program by a central processing unit so as to run a radiation measurement module, a signal measurement module, and a signal control module;
running the signal control module having steps of:
initiating a motor, a first electromagnetic valve, a second electromagnetic valve, and a third electromagnetic valve for transporting the ^{99m}Tc pertechnetate from the first container into a cation-exchange solid phase extraction chromatography column, an anion exchange column, and a waste bottle through a first pipeline, a second pipeline, a fourth pipeline, and a fifth pipeline; and
turning off the first electromagnetic valve and transporting the normal saline into the anion exchange column and a first receiving flask;
running the radiation measurement module through a first Geiger-Muller Counter to monitor activity; and
running he signal measurement module to weight the ^{99m}Tc pertechnetate inside the first receiving flask through a weighting scale member so as to check whether to interrupt the automatic control program or not.

11. The method as claimed in claim 10, wherein before the step of initiating a motor, a first electromagnetic valve, a second electromagnetic valve, and a third electromagnetic valve, the method further comprising a step of: initiating the motor, the second electromagnetic valve, the third electromagnetic valve and the fourth electromagnetic valve so as to transport sterilized water inside a third container into the waste bottle through a sixth pipeline, the second pipeline, the fourth pipeline, and the fifth pipeline.

12. The method as claimed in claim 10, wherein after the step of turning off the first electromagnetic valve and transporting the normal saline into the anion exchange column and a first receiving flask, the method further comprising a step of:
turning on a fifth electromagnetic valve for sending normal saline inside a fourth container into the anion exchange column through a seventh pipeline, and into a second receiving flask through the fourth pipeline.

13. The method as claimed in claim 10, wherein when turning off the first electromagnetic valve and transporting the normal saline into the anion exchange column and a first receiving flask, the method further having a step of: running filtering process after the normal saline passing through the anion exchange column and receiving the normal saline being filtered by the first receiving flask.
